(19) United States
(12) Patent Application Publication
(10) Pub. No.: US 2007/0148656 A1
(43) Pub. Date: Jun. 28, 2007

(54) Method for detecting the risk of cancer, coronary heart disease, and stroke by analysing a catalase gene

(76) Inventors: Jukka T. Salonen, Kuopio (FI); Jari Kalkkonen, Kuopio (FI); Marja Marchesani, Helsinki (FI); Mia Pirskanen, Kuopio (FI); Tomi-Pekka Tuomainen, Kuopio (FI); Pekka Uimari, Kuopio (FI); Teemu Vilppo, Kuopio (FI)

Correspondence Address: BIRCH STEWART KOLASCH & BIRCH, PO BOX 747, FALLS CHURCH, VA, 22040-0747, US

(21) Appl. No.: 10/589,364

(22) PCT Filed: Feb. 11, 2005
(86) PCT No.: PCT/FI05/50031

§ 371 Date: Feb. 27, 2007

(30) Foreign Application Priority Data
Feb. 13, 2004 (FI) 20040227

(51) Int. Cl. C12Q 1/68

(52) U.S. Cl. 435/6

(57) ABSTRACT

The present invention relates to variants in the catalase gene. The invention provides a method of identifying subject's susceptibility or predisposition to or risk of developing cancer, cancer death, CHD, and/or cerebrovascular stroke by detecting gene polymorphisms and other gene mutations in a biological sample from the subject. The invention also relates to a test kit and software for accomplishing the method. In addition, the invention provides a method for treating or preventing a human or animal suffering from cancer, CHD or cerebrovascular stroke, said method comprising a therapy enhancing catalase availability, production or concentration of the human subject or animal.

### FIELD OF THE INVENTION

The present invention relates to the use of catalase (EC 1.11.1.6) polymorphisms in detecting or predicting the risk of, or predisposition to cancer, cancer death, coronary heart disease (CHD), and stroke in a subject, as well as to a kit or assay for carrying out said method. This invention also relates to targeting catalase enhancing treatments in cancer, CHD, and stroke.

### BACKGROUND OF THE INVENTION

An excess of reactive oxygen species (ROS) contributes to the aging process and degenerative diseases, such as cardiovascular disease. Oxidative stress can also lead to DNA damage following carcinogenesis.¹ Catalase (EC 1.11.1.6) is an important antioxidative enzyme that detoxifies H₂O₂ into oxygen and water at a high rate, preventing harmful effects of ROS.¹ The mammalian catalase (˜240, 000 daltons) occurs as a complex of four identical subunits.² Together with superoxide dismutases (SODs) and glutathione peroxidases (GPXs), it forms the primary defense against oxidative stress in the human body.

On the basis of cell culture and animal experiments, excess H₂O₂ and lipid hydroperoxide concentration can lead to DNA damage resulting in cancer, and H₂O₂ scavengers and eliminators, such as excess intravenously infused catalase, can limit these damages.³⁻⁴ Urinary hydrogen peroxide levels have been lower in healthy controls, as compared with cancer patients.⁵ In most cancer cells, the catalase activity is low.⁶ For example, in lung cancer patients, catalase activity has been decreased in tumors, as compared with adjacent tumor-free lung tissues.⁷ In addition, there is some evidence that in cancer patients with advanced disease, high H₂O₂ content, formed as a result of tumor-induced granulocyte activation, could suppress the adaptive immune functions leading to further accelerated disease progression.⁸

It has been reported that platelet catalase activity is significantly lower in patients with CHD, as compared with healthy controls.⁹ Secondly, it has been found that healthy children with family history of early CHD have lower erythrocyte catalase activity than a control group of children with no family history of CHD.¹⁰

Genetic polymorphisms can attenuate the activity of catalase in tissues. The human catalase gene (CAT) consists of 13 exons and is located in chromosome 11p13¹¹. Previously, only rare mutations have been reported in the catalase gene, most of them being associated with acatalasemia, a disease in which erythrocyte catalase activity is low.^{2,12} Recently, two common promoter area SNPs have been found in positions 5′UTR-844 and -262 of the catalase gene.^{12,13} Of these two, the SNP in position-262 is located in the region important in the regulation of catalase gene expression.¹⁴

The publications and other material used herein to illuminate the background of the invention are incorporated herein by reference.

### SUMMARY OF THE INVENTION

The object of the present invention is a method of identifying risk of developing cancer (especially colon and rectal cancer), increased risk of cancer death, increased risk of prevalent CHD, and/or stroke by detecting catalase polymorphisms from a biological sample of a subject, such as a human. The information obtained from this method can be combined with other information concerning individuals, e.g. results from blood measurements, clinical examinations and questionnaires. The blood measurements may include the determination of blood or plasma or serum analytes such as serum ferritin and vitamin E content. The information to be collected by questionnaire may include information concerning age, family and medical history, and health-related habits such as smoking. These and further objects will be evident from the following description and claims.

Specifically, such a method comprises the steps of
a) providing a biological sample of the subject to be tested, and
b) detecting the presence or absence of specific variations in a catalase gene in the biological sample, the presence of a single copy or two copies of a specific variant indicating an increased risk of cancer, cancer deaths, coronary heart disease, and/or stroke in said subject.

### DETAILED DESCRIPTION OF THE PREFERRED EMBODIMENTS OF THE INVENTION

The present invention provides means for prognostic or diagnostic assays for determining if a subject is likely to develop cancer, coronary heart disease (CHD), and/or stroke, which is/are associated with the variation or dysfunction of a catalase gene. Basically, such assays comprise a detection step, wherein the presence or absence of a genetic alteration or defect in the catalase gene is determined in a biological sample taken from the subject. Said detection step can be performed, e.g., by methods involving sequence analysis, nucleic acid hybridisation, primer extension, restriction enzyme site mapping or antibody binding. These methods are well-known in the art (see, for example, Current Protocols in Molecular Biology, eds. Ausubel et al, John Wiley & Sons:1992).

In particular, the present invention is directed to a method of determining the presence or absence of a catalase polymorphism in a biological sample from a human for assessing the predisposition of an individual to cancer, coronary heart disease (CHD), and/or stroke. Said method comprises determining the sequence of the nucleic acid of a human at one or more of the positions (shown in Table 2) in the catalase gene or mRNA and determining the status of the human by reference to polymorphism in catalase gene. However, a person skilled in the art may carry out various polymorphism discovery methods to find other functional catalase gene mutations for use in the method of the invention. Such variants are deemed to be within the scope of the present invention from the teachings herein.

Numerous genotyping methods have been described in the art for analysing nucleic acids for the presence of specific sequence variations e.g. SNPs, insertions and deletions (for review see Syvänen, 1999, Human Mutation 13:1-10). In these methods a sample containing nucleic acid (e.g. blood, tissue biopsy or buccal cells) is obtained from the patient and the sequence variations of interest are identified and assessed from the nucleic acids.

Allelic variants in genes can be discriminated by enzymatic methods (with the aid of restriction endonucleases, DNA polymerases, ligases etc.), by electrophoretic methods (e.g. single strand conformation polymorphism (SSCP), heteroduplex analysis, fragment analysis and DNA sequencing), by solid-phase assays (dot blots, microarrays, microparticles, microtiter plates etc.) and by physical methods (e.g. hybridisation analysis, mass spectrometry and denaturing high performance liquid chromatography (DHPLC)). In most of the genotyping assays different polymerase chain reaction (PCR) applications are used both to increase the signal to noise ratio as well as spare sample nucleic acid before allele discrimination. Detectable labels (fluorochromes, radioactive labels, biotin, modified nucleotides, haptens etc) can be used to enhance visualization of allelic variants.

In a preferred embodiment of the invention a biological sample is contacted with oligonucleotide primers so that the nucleic acid region containing the potential single nucleotide polymorphism is amplified by polymerase chain reaction prior to determining the sequence. The final results can be obtained by using a method selected from, e.g., allele specific nucleic acid amplification, allele specific nucleic acid hybridisation (e.g. with a capturing probe), oligonucleotide ligation assay or restriction fragment length polymorphism (RFLP). These methods are well-known for a skilled person of the art (see, for example, Current Protocols in Molecular Biology, eds. Ausubel et al, John Wiley & Sons:1992, or Landegren et al, “Reading Bits of Genetic Information: Methods for Single-Nucleotide Polymorphism Analysis”, Genome Research 8:769-776).

The detection step of the method can also be a specific DNA-assay, such as a gene or DNA chip, microarray, strip, panel or similar combination of more than one genes, mutations or RNA expressions to be assayed.

The biological sample for the method can be, e.g., a blood sample or buccal swab sample. From said sample genomic DNA is isolated.

The subject to be tested is preferably a mammal, more preferably a primate, and most preferably a human.

The polymorphic sites can be analyzed individually or in sets for prognostic purposes. The conclusion drawn from the analysis depends on the nature and number of polymorphic sites analyzed. Some polymorphic sites have variant polymorphic forms that are causative of disease. Detection of such a polymorphic form provides at least a strong indication of presence or susceptibility to disease. Other polymorphic sites have variant polymorphic forms that are not causative of disease but are in equilibrium dislinkage with a polymorphic form that is causative. Thus, detection of noncausative polymorphic forms may also indirectly provide an indication of risk of presence or susceptibility to disease. Preferably, multiple variant forms at several polymorphic sites in catalase gene are detected to provide an indication of increased risk of presence or susceptibility to disease. The results from analyzing the polymorphic sites of the invention can be combined with analysis of other loci that associate with the same disease (i.e., cancer, prevalent CHD or stroke). Alternatively or additionally, the risk of disease can be confirmed by performing conventional medical diagnostic tests of patient symptoms.

In one preferred embodiment, the invention comprises the combination of information from a large number of variables (measurements) to predict susceptibility to cancer (especially to colorectal cancer), cancer death, CHD, and/or stroke. The predictor information includes an assessment of genotypes in genomic DNA and optionally data obtainable by interviews, questionnaires, clinical examination and/or blood analyte measurements.

Information concerning genomic DNA genotypes concerns polymorphisms such as single nucleotide polymorphisms (SNPs) and mutations in e.g. catalase. The data that can be obtained by interviews, questionnaires, clinical examination and/or blood analyte measurements includes information concerning such as:
1. Age
2. Smoking
3. Cancer history
4. Blood leukocyte count
5. Drug for high cholesterol
6. Serum ferritin
7. Serum vitamin E
8. Existing IHD disease
9. Diabetes mellitus, type 2
10. Retinol intake
11. Examination year
12. Drug for hypertension
13. Adulthood socio-economic status (SES)
14. Hypertension, HT
15. Ischemic heart disease (IHD) in family
16. Plasma fibrinogen
17. Hair mercury content
18. Serum triglycerides

In one specific embodiment, the invention is based on the principle that a small number of genotyping is performed. Any method to genotype mutations or other type of polymorphisms in a genomic DNA sample can be used. The score that predicts the probability of cancer, cancer death, prevalent CHD and/or stroke may be calculated using a multivariate failure time model or a logistic regression model:

Probability of cancer, cancer death, prevalent CHD or stroke=[1+e^{(-(-a+ε(bi*Xi))}]⁻¹, wherein e is Napier's constant, Xᵢ are variables related to preeclampsia, bᵢ are coefficients of these variables in the logistic function, and a is the constant term in the logistic function. The model may additionally include any interaction (product) or terms of any variables Xᵢ, e.g. bᵢXᵢ. Alternative statistical models are a failure-time models such as the Cox's proportional hazards' model and neural networking models.

The present invention also provides a method for treating or targeting the treatment of cancer, prevalent CHD or stroke in a subject with the disease by determining the pattern of alleles encoding a variant catalase gene, i.e. by determining if said subject's genotype of catalase gene is of the variant type, comprising the steps presented in the above detection method, and treating a subject of the variant genotype with a drug affecting catalase production or metabolism of the subject. The treatment may comprise a therapy which enhances catalase availability, production or concentration in the circulation of the human subject or animal. Such treatment can be a dietary treatment, a vaccination, gene therapy or gene transfer (see e.g. U.S. Pat. No. 6,627,615). Gene therapy is carried out, e.g., by transferring a non-variant catalase gene or fragment or derivative thereof.

It is further noted that catalase nucleic acid molecules, catalase polypeptides, catalase agonists, catalase antagonists, and derivatives, fragments, analogs and homologs thereof, can be incorporated into pharmaceutical compositions for the treatment according to the invention.

The invention also features prognostic kits for use in detecting the presence of catalase polymorphism in a biological sample. The kit provides means for assessing the predisposition of an individual to cancer, prevalent CHD and/or stroke mediated by variation or dysfunction of catalase. The kit can comprise a labelled compound capable of detecting catalase polypeptide or nucleic acid (e.g. mRNA) in a biological sample. The kit can also comprise nucleic acid primers or probes capable of hybridising specifically to at least of portion of a catalase gene or allelic variant thereof. The kit can be packaged in a suitable container and preferably it contains instructions for using the kit and optionally software to interpret the results of the detection.

The kit can be based on a capturing nucleic acid probe specifically binding to the variant genotype as defined in the invention, and/or on a DNA chip, microarray, DNA strip, DNA panel or real-time PCR based tests.

Furthermore, we have identified a novel variant form (SEQ ID NO: 26) of the human catalase (CAT) gene (SEQ ID NO: 24). This variant gene encodes a protein (SEQ ID NO: 27) with a substitution in the amino acid 316 of the polypeptide. Thus, preferably the presence or absence of Leu316Pro (T>C) mutation in Exon 8 of the catalase gene is detected in the method of the invention.

Nucleic acids which encode variant catalase, preferably from non-human species, such as murine or rat protein, can be used to generate either transgenic animals or “knock out” animals which, in turn, are useful in the development and screening of therapeutically useful reagents. A transgenic animal (e.g., a mouse) is an animal having cells that contain a transgene, which transgene was introduced into the animal or an ancestor of the animal at a prenatal, e.g., an embryonic, stage. A transgene is a DNA which is integrated into the genome of a cell from which a transgenic animal develops. In one embodiment, the human and/or mouse cDNA encoding variant catalase, or an appropriate sequence thereof, can be used to clone genomic DNA encoding variant catalase in accordance with established techniques and the genomic sequences used to generate transgenic animals that contain cells which express DNA encoding variant catalase. Methods for generating transgenic animals, particularly animals such as mice, have become conventional in the art and are described, for example, in U.S. Pat. Nos. 4,736,866 and 4,870,009.

Although particular embodiments have been disclosed herein in detail, this has been done by way of example for purposes of illustration only, and is not intended to be limiting with respect to the scope of the appended claims that follow. In particular, it is contemplated by the inventors that various substitutions, alterations, and modifications may be made to the invention without departing from the spirit and scope of the invention as defined by the claims. Thus, the described embodiments are illustrative and should not be construed as restrictive.

### EXPERIMENTAL SECTION

For the identification of the specific known SNPs mentioned in the experimental section we have used rs-identification numbers from the NCBI SNP database (http://www.ncbi.nlm.nih.gov/SNP/).

Sequencing of the Human Catalase Gene:

We sequenced all 13 exons and their 5-prime and the 3-prime flanking areas of the human catalase (CAT) gene in order to find sequence variants which could be linked with altered activity of the catalase enzyme. The material that we used included 25 samples from patients with low catalase enzyme activity (15.9-26.7) and 25 samples with high catalase enzyme activity (53.5-71.7). The nucleotide sequence of the primer pair for the amplification of human CAT gene exons (and the subsequent flanking intron 5′ and 3′ areas) are presented in Table 1. The primers are designed so that they amplify parts of the 5-prime and the 3-prime flanking areas of the target exon. The CAT gene exons 3 and 4, exons 5 and 6, exons 7 and 8, and exons 12 and 13 were amplified in the same PCR fragment.

TABLE 1

Nucleotide sequences of the primer pairs for
the amplification of human CAT gene exons 1-13.

| | | |
|---|---|---|
| Amplified | | Annealing |
| CAT exon | PCR primer nucleotide sequences | temperature |

| | | | |
|---|---|---|---|
| exon 1 | 5′- gtc taa gta ttc cgt ctg c -3′ | (SEQ ID NO:1) | 58° C. |
| | 5′- cct gct tcg gcg aat gta -3′ | (SEQ ID NO:2) | |
| exon 2 | 5′- gct atg tac ccg tga cag -3′ | (SEQ ID NO:3) | 59° C. |
| | 5′- aac act tga ccc agg tgc -3′ | (SEQ ID NO:4) | |
| exons 3-4 | 5′- gtc tca tgg taa gga ttt ctg -3′ | (SEQ ID NO:5) | 56° C. |
| | 5′- agt cca gac aac tcg cat tc -3′ | (SEQ ID NO:6) | |
| exons 5-6 | 5′- gtg gac tga att agc tgg tgg -3′ | (SEQ ID NO:7) | 59° C. |
| | 5′- gag gca taa tta aac act gca tc -3′ | (SEQ ID NO:8) | |
| exons 7-8 | 5′- gtg tta ctc ata atc ctt caa t -3′ | (SEQ ID NO:9) | 54° C. |
| | 5′- gtc ttc aca tat gta ggg atc -3′ | (SEQ ID NO:10) | |
| exon 9 | 5′- gta acc atg tac aga gtg c -3′ | (SEQ ID NO:11) | 51° C. |
| | 5′- agg agg tcc tgc ggg gc -3′ | (SEQ ID NO:12) | |
| exon 10 | 5′- gag att cat tca taa agt gcg -3′ | (SEQ ID NO:13) | 59° C. |
| | 5′- gtg act tcc ata gca gat aaa g -3′ | (SEQ ID NO:14) | |
| exon 11 | 5′- cta agt gtt gta gta ggt gaa -3′ | (SEQ ID NO:15) | 57° C. |
| | 5′- acg atg gat atg cca gac cag -3′ | (SEQ ID NO:16) | |
| exons | 5′- gag tga tat agt agg gag tta g -3′ | (SEQ ID NO:17) | 56° C. |
| 12-13 | 5′- tta aca tta atg taa ctc cag tg -3′ | (SEQ ID NO:18) | |

The PCR amplification was conducted in a 30 μl volume: the reaction mixture contained 60 ng human genomic DNA (extracted from peripheral blood), 1×PCR Buffer (1.5 mM MgCl₂, QIAGEN), 100 μM of each of the nucleotides (dATP, dCTP, dGTP, dTTP), 15 pmol of each of the primers, 1.25 unit of the DNA polymerase (QIAGEN, Hot Start Taq DNA polymerase).

The target DNA sequences (exons 1-13 of the CAT gene) were amplified in the above mentioned PCR reaction by using the PTC-220 DNA Engine Dyad PCR machine (MJ Research) with the PCR program conditions as follows: first the reaction was hold 10 minutes at 95° C., then the following three steps were repeated for 35 times: 45 seconds at 94° C., 30 seconds at annealing temperature (see table 1), 1 minute at 72° C. after which the reaction was kept at 72° C. for 5 minutes, and finally hold at 4° C.

Before the sequencing reaction the amplified CAT gene exon PCR products were purified with the GFX™96 PCR Purification Kit (Amersham Pharmacia Biotech Inc, Piscataway, N.J.). The sequencing reactions were made by using the BigDye™ Terminator Cycle Sequencing v2.0 Ready Reactions with AmpliTaq® DNA Polymerase, FS DNA Sequencing Kit (Applied Biosystems, Foster City, Calif.).

Cycle sequencing was made in the PTC-220 DNA Engine Dyad PCR machine (MJ Research) with the program as follows: the following three steps were repeated for 25 cycles; 10 seconds at 96° C., 5 seconds at 50° C. and 4 minutes at 60° C. after which the reaction hold at 4° C. To perform cycle sequencing under standard conditions refer to ABI PRISM® 3100 Genetic Analyzer Sequencing Chemistry Guide, Applied Biosystems, Foster City, Calif.

Dye terminator removal and sequencing reaction clean up was made by using the MultiScreen®-HV filtration plate (Millipore, Bedford, Mass.). After the clean up the samples were transferred to MicroAmp® Optical 96-Well Reaction Plate (Applied Biosystems, Foster City, Calif.) and sequenced by using the ABI PRISM® 3100 Genetic Analyzer (Applied Biosystems, Foster City, Calif.), which is an automated fluorescence-based capillary electrophoresis DNA analysis system with 16 capillaries.

In sequencing of the 13 CAT gene exons we found five different DNA variants (table 2). Of the five DNA variants one was previously unknown i.e. CAT Exon 8 Leu316Pro T>C mutation. The other four DNA variants in the table have already been identified and their NCBI SNP database (http://www.ncbi.nlm.nih.gov/SNP/) rs-identification numbers are given in the table 2.

TABLE 2
The found CAT gene sequence variants
and their identification numbers.

| | NCBI SNP database |
|---|---|
| CAT gene variant site | identification number |
| CAT 5′UTR −262 C > T | (rs1001179) |
| CAT 5′UTR −21 T > A | (rs7943316) |
| CAT 5′UTR 49 C > T | (rs1049982) |
| CAT Exon 8 Leu316Pro T > C | Previously unknown CAT |
| | gene mutation |
| CAT Exon 9 Asp389Asp C > T | (rs769217) |

Genotyping of the Human Catalase Gene Variants:

Genotypings were conducted among the subjects of the KIHD cohort with Snapshot method (Applied Biosystems). In a snapshot reaction the genomic DNA region containing the variation in question is amplified with PCR. The amplified PCR product is purified and used as a template in the snapshot reaction. For the snapshot reaction an extension primer is designed so that the 3′ end of the primer is immediately adjacent to the polymorphic site of interest. In the snapshot reaction the extension primer hybridizes to its complementary template in the presence of fluorescent labelled dideoxy-NTPs ([F]ddNTPs) and DNA polymerase. The polymerase extends the primer by only one nucleotide, adding a single [F]ddNTP to its 3′ end. Because each of the four [F]ddNTPs are labeled with different fluorecent dyes the individual genotypes are detectable after electrophoresis with ABI Prism 3100 Genetic Analyzer (Applied Biosystems). Electrophoresis data is processed and the genotypes are visualized by using the GeneScan Analysis version 3.7 (Applied Biosystems).

When multiple SNPs are determined in the same reaction, the extension primers need to differ significantly in length (4-6 nucleotides) to avoid overlap between the final SNaPshot products. This can be accomplished by adding a variable number of nucleotides dT, dA, dC or cGATC to the 5′ end of the different extension primers. The different SNPs can then be detected in the capillary electrophoresis according to the different size of the SNaPshot product. To perform SnaPshot genotyping under standard conditions, refer to the user manual (ABI Prism SnaPshot Multiplex kit, Protocol, Applied Biosystems).

The genomic DNA regions containing the mutations in question were amplified all in one single reaction mix (i.e. multiplex PCR) with PTC-220 DNA Engine Dyad PCR machine (MJ Research). The PCR amplification was conducted in a 30 μl volume: the reaction mixture contained 60 ng human genomic DNA (extracted from peripheral blood), 1×PCR Buffer (QIAGEN), 200 μM of each of the nucleotides (DATP, dCTP, dGTP, dTTP), 10-20 pmol of each of the PCR primers and 1.25 units of the DNA polymerase (QIAGEN, Hot Start Taq DNA polymerase). The PCR protocol was as follows: first the reaction was hold 10 minutes at 95° C., then the following three steps were repeated for 35 cycles: 30 seconds at 94° C., 45 seconds at 53° C., 1 minute at 72° C., after which the reaction was kept at 72° C. for an additional 5 minutes and finally hold at 4° C.

The nucleotide sequence of the primer pair for the amplification of human Catalase gene (CAT) CAT 5′UTR-262 C>T, CAT 5′UTR-21 T>A and CAT 5′UTR 49 C>T variants was as follow: 5′-GTC TAA GTA TTC CGT CTG C-3′ (SEQ ID NO:1) and 5′-CCT GCT TCG GCG AAT GTA-3′ (SEQ ID NO:2).

The nucleotide sequence of the primer pair for the amplification of human catalase gene (CAT) exon 8 Leu316Pro T>C mutation was as follow: 5′-GTG TTA CTC ATA ATC CTT CAA T-3′ (SEQ ID NO:9) and 5′-GTC TTC ACA TAT GTA GGG ATC-3′ (SEQ ID NO:10).

The nucleotide sequence of the primer pair for the amplification of human catalase gene (CAT) exon 9 Asp389Asp C>T (rs769217) mutation was as follow: 5′-GTA ACC ATG TAC AGA GTG C-3′ (SEQ ID NO:11) and 5′-AGG AGG TCC TGC GGG GC-3′ (SEQ ID NO:12).

The PCR products were purified with SAP (Shrimp Alkaline Phosphatase, USB) and ExoI (Exonuclease I, New England Biolabs) treatment. This was done to avoid the participation of the unincorporated dNTPs and primers from the PCR reaction to the subsequent primer-extension reaction. More specifically, 2.5 μl of SAP (1 unit/μl), 0.25 μl of ExoI (20 units/μl), 1.0 μl of 10× ExoI buffer (New England Biolabs) and 6.25 μl H₂O were added to 5 μl of the PCR product. Reaction was mixed and incubated at 37° C. for 1 hour, at 75° C. for 15 minutes and stored at 4° C. In the subsequent primer extension reaction (SNaPshot reaction) 5 μl of SNaPshot Multiplex Ready Reaction Mix (Applied Biosystems), 3 μl of purified PCR products, 1 μl of pooled extension primers (depending of the signal in the SNaPshot reaction, the primer concentrations in the mix can range between 0.05 μM and 1 μM) and 1 μl water are mixed in a tube. The reaction is incubated at 94° C. for 2 minutes and then subject to 25 cycles of 95° C. for 5 s, 50° C. for 5 s and 60° C. for 5 s in a PTC-220 DNA Engine Dyad PCR machine (MJ Research). After the primer extension reaction 1 unit of SAP was added to the reaction mix and the reaction was incubated at 37° C. for 1 hour, at 75° C. for 15 minutes and kept at 4° C.

The nucleotide sequence of the extension primer for the genotyping of human CAT 5′UTR-262 C>T (rs1001179) variant in a SNaPShot reaction was 5′-TTT TTT TTT TTT TTC GCC CTG GGT TCG GCT AT-3′ (SEQ ID NO:19).

The nucleotide sequence of the extension primer for the genotyping of human CAT 5′UTR-21 T>A (rs7943316) variant in a SNaPShot reaction was 5′-TTT TTT TTT TTT TTT TTT GAG CCT GAA GTC GCC ACG G-3′ (SEQ ID NO:20).

The nucleotide sequence of the extension primer for the genotyping of human CAT 5′UTR 49 C>T (rs1049982) variant in a SNaPShot reaction was 5′-TTT TTT TTT TTT TTT TTT TTT TTG AGG CCT CCT GCA GTG TTC-3′ (SEQ ID NO:21).

The nucleotide sequence of the extension primer for the genotyping of human CAT exon 8 c.946T>C Leu316Pro variant in a SNaPShot reaction was 5′-TTT TTT TTT TTT TTT TTT TTT TTT TTT TCT CAT CCC AGT TGG TAA AC-3′ (SEQ ID NO:22).

The nucleotide sequence of the extension primer for the genotyping of human CAT exon 9 c.1167C>T, Asp389Asp (rs769217) variant in a SNaPShot reaction was 5′-TTT TTT TTT TTT TTTTTT TTT TTT TTT TTT TTT TGG CCA ACT ACC AGC GTG A-3′ (SEQ ID NO:23).

Aliquots of 1 μl of pooled SNaPshot products, 9.00 μl of Hi-Di formamide (Applied Biosystems) and 0.25 μl GeneScan-120 LIZ size standard (Applied Biosystems) were combined in a 96-well 3100 optical microamp plate (Applied Biosystems). The reactions were denatured by placing them at 95° C. for 5 minutes and then loaded onto a ABI Prism 3100 Genetic Analyzer (Applied Biosystems). Electrophoresis data was processed and the genotypes were visualized by using the GeneScan Analysis version 3.7 (Applied Biosystems).

Measurement of Blood Catalase Activity

The blood catalase activity was measured for 546 men at the KIDH 11-year follow-up from fasting whole blood. Catalase decomposes hydrogen peroxide to less harmful oxygen and water. The measurement method for catalase activity was based on the competition between sample catalase activity and the simultaneous colour forming reaction.¹⁵ Uric acid was used to buffer H₂O₂ concentration in a reaction catalyzed by uricase (EC 1.7.3.3). Catalase activity was measured by the competitive enzymatic color reaction, where horseradish peroxidase (EC 1.11.1.7)/Trinder reagent, as color forming reagent, competed simultaneously with catalase of the availability/sufficiency of H₂O₂. Percentual inhibitions for standards and samples were calculated against a blank reaction. Commercial catalase enzyme (Sigma, St. Louis, Mo.), whose activity was checked according to manufacturer instructions, was used to obtain a standard curve. Activities were measured using an auto-analyzer (Konelab 20, Thermo Electron Corporation, Vantaa, Finland).

Ascertainment of Cancers, Deaths and Strokes

Our study cohort was record-linked with the cancer registry¹⁶ data by using the unique personal identification code (social security number) that all Finns have. Cancer history before the baseline examination was recorded by a self-assessment questionnaire. Deaths were ascertained by a computer linkage to the national death registry using the Finnish social security number. There were no losses to follow-up. All deaths that occurred from the study entry to Dec. 31, 2001, were included. Deaths were coded according to the International Classification of Diseases (9th ed.; ICD-9).¹⁷ Follow-up data concerning strokes were registered as part of the multinational MONICA Project, and by computerized linkage to the Finnish national hospital discharge registry and death certificate registers.

Questionnaires

The history and the family history of coronary heart disease (CHD, IHD), and smoking were recorded using a self-assessment questionnaire, checked by an interviewer.¹⁸ Interviews to obtain medical history were conducted by a physician. Food and nutrient consumption was assessed by a nutritionist—instructed 4-day food recording by household measures.¹⁹ Socio-economic status was measured with a summary index that combined income, education, occupation, occupational prestige, material standard of living, and housing conditions.²⁰ Diabetes was defined as fasting blood glucose >6.7 mmol/l or if a subject had medication for diabetes.

Other Measurements

Subjects were instructed to fast overnight (12 hours) and abstain from smoking for 12 hours and from drinking alcohol for three days prior to the visit. The brachial venous blood samples were drawn with vacuum tubes from a subject after a 30-minute rest in a supine position. No tourniquet was used. Chemical measurements such as serum ferritin,²¹ and serum lipid-standardized vitamin E,²² were carried out as described in detail elsewhere. Blood leukocyte count was assessed by a cell counter (Coulter Counter Electronics, Luton, England). Plasma fibrinogen levels were measured on the basis of clotting of diluted plasma with excess thrombin (Coagulometer KC4, Heinrich Amelung, Lemgo, Germany). Serum triglycerides were determined with a commercial kit (Boehringer Mannheim, Mannheim, Germany) using an auto-analyser. Hair mercury content was assessed as previously described in detail.²³

Statistical Analysis

A one-way analysis of variance (ANOVA) test was used to assess the heterogeneity in variables between genotypes. Relative risks were estimated as relative hazards, the antilog of the partial coefficient, using the Cox proportional hazards model. All data analyses were carried out using SPSS for Windows (version 11.01, SPSS Inc., Chigaco, Ill.). A two-sided P<0.05 was considered statistically significant in all comparisons.

Testing the Risk of Cancer, Cancer Death, Stroke and Prevalent CHD:

Catalase 5′UTR-262 polymorphism was determined in 1,593 Eastern Finland men that belong to the cohort of the “Kuopio Ischaemic Heart Disease Risk Factor Study” (KIHD), a population study to investigate genetic and other risk factors for cardiovascular diseases, cancers and deaths.¹⁸ Of these 1,593 men, 153 developed cancer and 97 suffered cerebrovascular stroke within a mean follow-up of 13.6 years, 48 men died of cancer and 203 of any cause within a mean follow-up time of 13.9 years, 326 had symptomatic CHD or had previous CHD history.

CAT 5′UTR-262 (C>T) Polymorphism:

There were 722 subjects (45.3%) with the CAT-262 CC genotype, 685 subjects (43.0%) with CT genotype, and 186 subjects (11.7%) with TT genotype in CAT 5′UTR-262. Of these men, blood catalase activity was determined for 546 men in connection with the 1′-year follow-up visit. Subjects with the TT genotype had 8.0% and, the subjects having TC genotype 7.3% lower activity, as compared to CC genotype (p<0.001). For this reason the statistical disease prediction models were formed to compare catalase −262 CC genotype to TT and TC genotypes.

In step-up Cox models, examination year, age, genotypes with T allele and the most important risk factors of the each outcome investigated were tested (p=0.05 for entry). Subjects with T allele had 1.52-fold (95% CI, 1.09 to 2.12, p=0.013) risk to develop cancer, as compared with CC genotype (Table 3). As other risk factors, age, smoking, positive cancer history, leukocytes, drug for high cholesterol, and serum ferritin, and as a protective factor, serum vitamin E entered into the model.

The T allele seemed to expose the strongest to the colorectal cancer, relative risk (RR) of 3.28 (95% CI, 1.09 to 9.92, p=0.035). In addition to the T allele, age, cancer history, existing ID disease, and diabetes mellitus type 2 entered as risk factors into the model.

Subjects with the T allele had a 3.10-fold (95% CI, 1.57 to 6.11, p=0.001) risk to suffer cancer death, as compared with the CC genotype. Of other risk factors, age, smoking, leukocytes and retinol intake entered into the model (Table 4).

Subjects with T allele had a 1.50-fold (95% CI, 1.14 to 1.97, p=0.004) risk to have prevalent CHD, as compared to CC genotype. Other risk factors were age, smoking, drug for high cholesterol, examination year, drug for hypertension, low adulthood socio-economic status (SES), hypertension, ischemic heart disease in family, high plasma fibrinogen, hair mercury content and serum triglyceride levels (Table 5).

CATLeu316Pro (7>C) Polymorphism:

There were 1575 subjects (98.9%) with TT genotype, and 18 subjects (1.1%) with the CT genotype. There were no CC homozygous subjects. Of these men, blood catalase activity was determined for 546 men in connection with the 11-year follow-up visit. Subjects with the TT genotype (n=536) had 31.2% higher blood catalase activity, as compared with the CT genotype (n=10) (p<0.001).

In a step-up Cox model, examination year, age, and all of the polymorphisms (except CAT-262) were offered for the model (p=0.05 for entry). CT heterozygous subjects (for CAT Leu316Pro (T>C) polymorphism) were at an increased risk of stroke, as compared with the TT homozygous subjects, RR=3.15 (95% CI 1.00 to 10.00, p=0.050). Also age entered into the model. A total of 3 strokes (16.7% incidence) occurred among CT heterozygous subjects, and there were 94 strokes (6.0% incidence) among TT homozygous subjects.

CATAsp389Asp C>T Polymorphism:

There were 1041 subjects with CC genotype, 473 subjects with the CT genotype and 79 subjects with the TT genotype. Of these men, blood catalase activity was determined for 546 men. Subjects with the TT genotype (n=21) had 5.6%, and subjects with TC genotype 3.5% (n=170) lower blood catalase activity, as compared with the CC genotype (n=355) (p=0.031 for the trend). After forcing for examination year and age, the T allele tended to increase both the risk of cancer (RR=1.09, 95% CI, 0.78 to 1.52, p=0.599) and the risk of stroke (RR=1.25, 95% CI, 0.83 to 1.88, p=0.289).

TABLE 3
T allele in position 5′UTR −262 and cancer incidence based on Cox regression model.

| | | | 95.0% CI for Exp(B) | | Statistical |
|---|---|---|---|---|---|
| | B | Exp(B) | Lower bound | significance | significance |
| Catalase 5′UTR −262 CT or TT | 0.4200 | 1.52 | 1.09 | 2.12 | 0.013 |
| (1 = yes vs. 0 = no) | | | | | |
| Age (years) | 0.1233 | 1.13 | 1.09 | 1.17 | <0.001 |
| Smoker (1 = yes vs. 0 = no) | 0.5327 | 1.70 | 1.20 | 2.43 | 0.003 |
| Drug for high cholesterol | 1.3465 | 3.84 | 1.19 | 12.44 | 0.025 |
| (yes = 1 vs. no = 0) | | | | | |
| Serum ferritin (μg/l) | 0.0009 | 1.00 | 1.00 | 1.00 | 0.015 |
| Blood leukocyte count (10⁹/l) | 0.1058 | 1.11 | 1.01 | 1.23 | 0.039 |
| Serum lipid-standardized | −1.0118 | 0.36 | 0.16 | 0.85 | 0.019 |
| vitamin E (μmol/l) | | | | | |
| Positive cancer history | 1.0966 | 2.99 | 1.39 | 6.46 | 0.005 |
| (1 = yes vs. 0 = no) | | | | | |

TABLE 4
T allele in position 5′UTR −262 and cancer mortality based on Cox regression model.

| | | | 95% CI ofr Exp(B) | | Statistical |
|---|---|---|---|---|---|
| | B | Exp(B) | Lower bound | Upper bound | significance |
| Catalase 5′UTR −262 CT or TT | 1.1302 | 3.10 | 1.57 | 6.11 | 0.001 |
| (yes = 1 vs. no = 0) | | | | | |
| Age (years) | 0.0920 | 1.10 | 1.03 | 1.16 | 0.003 |
| Smoker (1 = yes vs. 0 = no) | 0.9028 | 2.47 | 1.34 | 4.55 | 0.004 |
| Blood leukocyte count (10⁹/l) | 0.2074 | 1.23 | 1.06 | 1.42 | 0.005 |
| Retinol intake (μg/day) | 0.0001 | 1.00 | 1.00 | 1.00 | 0.024 |

TABLE 5
T allele in position 5′UTR −262 and prevalent CHD based on logistic regression model.

| | | | 95.0% CI for Exp(B) | | Statistical |
|---|---|---|---|---|---|
| | B | Exp(B) | Lower bound | Upper bound | significance |
| Catalase 5′UTR −262 CT or TT | 0.4039 | 1.50 | 1.14 | 1.97 | 0.004 |
| (yes = 1 vs. no = 0) | | | | | |
| Age (years) | 0.0515 | 1.05 | 1.03 | 1.08 | <0.001 |
| Examination year | 0.1073 | 1.11 | 1.02 | 1.22 | 0.016 |
| Smoker (1 = yes vs. 0 = no) | 0.3249 | 1.38 | 1.02 | 1.87 | 0.035 |
| Drug for high cholesterol | | | | | |
| (1 = yes vs. 0 = no) | 1.3128 | 3.72 | 0.88 | 15.73 | 0.074 |
| Drug for hypertension | 1.0944 | 2.99 | 2.05 | 4.35 | <0.001 |
| (1 = yes vs. 0 = no) | | | | | |
| Low adulthood socioeconomic status, SES | 0.0884 | 1.09 | 1.05 | 1.13 | <0.001 |
| Hypertension (1 = yes vs. 0 = no) | 0.2935 | 1.34 | 0.95 | 1.90 | 0.097 |
| Ischemic heart disease in family | 0.4205 | 1.52 | 1.16 | 2.00 | 0.002 |
| (1 = yes vs. 0 = no) | | | | | |
| Plasma fibrinogen (g/l) | 0.2172 | 1.24 | 0.96 | 1.61 | 0.099 |
| Hair mercury content (μg/g) | 0.1455 | 1.16 | 1.08 | 1.24 | <0.001 |
| Serum triglycerides (mmol/l) | 0.1440 | 1.15 | 0.99 | 1.35 | 0.066 |

### REFERENCES

1. Halliwell B, Gutterridge J M C. Free Radicals in biology and medicine. Oxford, Clarendon Press, 1989.
2. Ogata M. Acatalasemia. Hum Genet 86, 331-340, 1991.
3. Kaneko T, Tahara S. Formation of 8-oxo-2′-deoxyguanosine in the DNA of human diploid fibroblasts by treatment with linoleic acid hydroperoxide and ferric ion. Lipids 35, 961-965, 2000.
4. Nishikawa M, Tamada A, Kumai H, Yamashita F, Hashida M. Inhibition of experimental pulmonary metastasis by controlling biodistribution of catalase in mice. Int J Cancer 99, 474-479, 2002.
5. Banerjee D, Madhusoodanan U K, Nayak S, Jacob J. Urinary hydrogen peroxide: a probable marker of oxidative stress in malignancy. Clin Chim Acta 334, 205-209, 2003.
6. Oberley T D, Oberley L W. Antioxidant enzyme levels in cancer. Histol Histopathol 12, 525-535, 1997.
7. Chung-man Ho J, Zheng S, Comhair S A, Farver C, Erzurum S C. Differential expression of manganese superoxide dismutase and catalase in lung cancer. Cancer Res 61, 8578-8585, 2001.
8. Schmielau J, Finn O J. Activated granulocytes and granulocyte-derived hydrogen peroxide are the underlying mechanism of suppression of t-cell function in advanced cancer patients. Cancer Res 61(12), 4756-4760, 2001.
9. Buczynski A, Wachowicz B, Kedziora-Kornatowska K, Tkaczewski W, Kedziora J. Changes in antioxidant enzymes activities, aggregability and malonyldialdehyde concentration in blood platelets from patients with coronary heart disease. Atherosclerosis 100, 223-228, 1993.
10. Siemianowicz K, Gminski J, Francuz T, Wojcik A, Posielezna B. Activity of antioxidant enzymes in children from families at high risk of premature coronary heart disease. Scand J Clin Lab Invest 63(2), 151-158, 2003.
11. Quan F, Korneluk R G, Tropak M B, Gravel R A. Isolation and characterization of the human catalase gene. Nucleic Acids Res 14, 5321-5335, 1986.
12. Forsberg L, Lyrenas L, de Faire U, Morgenstern R. A common functional C-T substitution polymorphism in the promoter region of the human catalase gene influences transcription factor binding, reporter gene transcription and is correlated to blood catalase levels. Free Radic Biol Med 30, 500-505, 2001.
13. Jiang Z, Akey J M, Shi J, Xiong M, Wang Y, Shen Y, Xu X, Chen H, Wu H, Xiao J, Lu D, Huang W, Jin L. A polymorphism in the promoter region of catalase is associated with blood pressure levels. Hum Genet 109, 95-98, 2001.
14. Nenoi M, Ichimura S, Mita K, Yukawa O, Cartwright I L. Regulation of the catalase gene promoter by SpI, CCAAT-recognizing factors, and a WT1/Egrrelated factor in hydrogen peroxide-resistant HP100 cells. Cancer Res 61, 5885-5894, 2001.
15. Slaughter M R, O'Brien P J. Fully-Automated Spectrophotometric Method of Measurement of Antioxidant Activity of Catalase. Clin Biochem 33, 525-534,
16. Teppo L, Pukkala E, Lehtonen M. Data quality and quality control of a population-based cancer registry. Experience in Finland. Acta Oncol 33, 365-369, 1994.
17. World Health Organization. International Classification of Diseases, 9th rev. (ICD-9). Geneva, Switzerland: WHO, 1977.
18. Salonen J T. Is there a continuing need for longitudinal epidemiologic research ? The Kuopio Ischaemic Heart Disease Risk Factor Study. Ann Clin Res 20, 46-50, 1988.
19. Ihanainen M, Salonen R, Seppiinen R, Salonen J T. Nutrition data collection in the Kuopio Ischaemic Heart Disease Risk Factor Study: Nutrient intake of middle-aged Eastern Finnish men. Nutr Res 9, 597-604, 1989.
20. Kaplan G A, Salonen J T. Socioeconomic conditions in childhood and ischaemic heart disease during middle age. BMJ 301, 1121-1123, 1990.
21. Tuomainen T P, Punnonen K, Nyyssonen K, Salonen J T: Association between body iron stores and the risk of acute myocardial infarction in men. Circulation 97, 1461-1466, 1998.
22. Porkkala-Sarataho E, Nyyssönen K, Salonen J T. Increased oxidation resistance of atherogenic plasma lipoproteins at high vitamin E levels in non-vitamin E supplemented men. Atherosclerosis 124, 83-94, 1996.
23. Salonen J T, Seppanen K, Nyyssönen K, Korpela H, Kauhanen J, Kantola M, Tuomilehto J, Esterbauer H, Tatzber F, Salonen R. Intake of mercury from fish, lipid peroxidation, and the risk of myocardial infarction and coronary, cardiovascular, and any death in eastern Finnish men. Circulation 91, 645-55, 1995.

1. A method for detecting genetic variation or polymorphism in a catalase gene comprising:
a) providing a biological sample taken from a subject to be tested; and
b) detecting the presence or absence of a variant genotype of the catalase gene in the biological sample, the presence of a variant catalase genotype indicating an increased risk or a susceptibility to cancer, colon cancer, rectal cancer, cancer death, coronary heart disease (CHD), and/or cerebrovascular stroke in said subject.

2. The method according to claim 1, wherein said variant genotype of the catalase gene is a homo- or heterozygote form of the mutation.

3. The method according to claim 1, wherein the detection step is a DNA-assay.

4. The method according to claim 1, wherein the detection step is carried out using a gene or DNA chip, microarray, strip, panel or similar combination of more than one genes, mutations, catalase RNA expressions or catalase concentration or activity to be assayed.

5. The method according to claim 1, wherein an allelic pattern is determined using polymerase chain reaction.

6. The method according to claim 1, wherein the biological sample is a blood sample or buccal swab sample.

7. The method according to claim 1, wherein the detection step is based on a capturing probe.

8. The method according to claim 1, wherein said method is used for determining whether a subject will benefit from treatment with a drug, nutrient or other therapy enhancing catalase production, levels or activity or inhibiting catalase catabolism or elimination in the subject.

9. The method according to claim 1, wherein said method is used for determining whether a subject will be at increased risk of adverse effects or reactions if catalase antagonists are administered to a subject.

10. The method according to claim 1, further comprising:
selecting a subject with a catalase gene sequence reducing the expression, production or levels of catalase enzyme for clinical drug trials testing the cancer, coronary heart disease and/or stroke preventing effects of compounds.

11. The method according to claim 1, wherein the detected mutation is −262 C>T of 5′ UTR of the catalase gene.

12. The method according to claim 1, wherein the detected mutation is Exon 8 Leu316Pro (T>C) of the catalase gene.

13. The method according to claim 1, wherein the detected mutation is Exon 9 Asp389Asp (C>T) of the catalase gene.

14. The method according to claim 1, further comprising:
combining information concerning age, smoking, cancer history, leukocytes, drug for high cholesterol, serum ferritin, serum vitamin E, existing IHD disease, diabetes mellitus type 2, and retinol intake, drug for hypertension, adulthood socio-economic status (SES), HT, ischemic heart disease in family, plasma fibrinogen, mercury from hair and serum triglycerides in blood of the subject with the results from step b) for confirming the indication obtained from said step.

15. The method according to claim 1, further comprising:
calculating the probability of cancer, cancer death, coronary heart disease (CHD), and/or cerebrovascular stroke using a logistic regression equation as follows:
Probability of a condition=[1+e^{-(a+ε(bi*Xi)]}⁻¹,
where e is Napier's constant, Xᵢ is a variable related to the cancer or cancer deaths, bᵢ is a coefficient of these variables in the logistic function, and a is the constant term.

16. The method according to claim 15, wherein a and bᵢ are determined in the population in which the method is to be used.

17. The method according to claim 15, wherein Xᵢ is selected from among the variables that have been measured in the population in which the method is to be used.

18. The method according to claim 15, wherein bᵢ are between the values of −20 and 20

19. The method according to claim 15, wherein Xᵢ's are between −99999 and 99999.

20. The method according to claim 15, wherein i are between the values 0 (none) and 100,000.

21. The method according to claim 15, wherein subject's short term, median term, and/or long term risk of cancer, CHD, and/or stroke is predicted.

22. A kit for detecting genetic variation or polymorphism in the catalase gene for the determination of a risk of cancer, especially colon and rectal cancer, cancer deaths, CHD, and/or stroke, in a subject, comprising means for catalase gene allele detection, and optionally software to interpret the results of the determination.

23. The kit according to claim 22 comprising a capturing nucleic acid probe specifically binding to the variant selected from at least one of the group consisting of: −262 C>T of 5′ UTR of the catalase gene, Exon 8 Leu316Pro (T>C) of the catalase gene and Exon 9 Asp389Asp (C>T) of the catalase gene.

24. The kit according to claim 22, comprising a DNA chip, microarray, DNA strip, DNA panel or real-time PCR based tests.

25. The kit according to claim 22, comprising a questionnaire for obtaining patient information concerning age, smoking, cancer history, drug for high cholesterol, existing IHD disease, diabetes mellitus type 2, and retinol intake, drug for hypertension, adulthood socioeconomic status (SES), HT, and ischemic heart disease in family.

26. An isolated variant nucleic acid encoding catalase protein, said nucleic acid comprising CAT Exon 8 Leu316Pro (T>C) mutation.

27. The nucleic acid according to claim 26 further comprising CAT −262 C>T 5′ UTR and/or CAT Exon 9 Asp389Asp (C>T) mutation.

28. The nucleic acid according to claim 26, wherein said nucleic acid is a genomic nucleotide sequence.

29. The nucleic acid according to claim 28, wherein said nucleic acid is cDNA.

30. The nucleic acid according to claim 26 comprising an RNA sequence.

31. The nucleic acid according to 26 having the nucleic acid sequence set forth in SEQ ID NO:26.

32. A capturing probe specifically binding to the nucleic acid according to claim 26.

33. The capturing probe according to claim 32, which comprises a single strand of the cDNA comprising the CAT Exon 8 Leu316Pro (T>C) mutation.

34. The capturing probe according to claim 32, which specifically binds to variant catalase nucleic acid comprising the CAT Exon 8 Leu316Pro (T>C) mutation, but does not bind to non-variant catalase.

35. A method for determining the presence or absence of a nucleic acid as defined in claim 26 in a biological sample comprising:
a) treating said sample to obtain single stranded target nucleic acid, or if the target nucleic acid are already single stranded, directly employing step (b);
b) contacting said target nucleic acid with a capturing nucleic acid probe and a detector nucleic acid probe; and
c) detecting the complex of capturing probe, target nucleic acid and detector probe.

36. The method according to claim 35, wherein the capturing nucleic acid probe is attached or capable of attaching to a solid phase, and comprises the cDNA sequence comprising the CAT Exon 8 Leu316Pro (T>C) mutation, and wherein a detected signal from the solid phase is an indication of the presence in the sample of a nucleic acid comprising the CAT Exon 8 Leu316Pro (T>C) mutation.

37. The method according to claim 35, wherein the capturing nucleic acid probe is attached or capable of attaching to a solid phase, and comprises a cDNA corresponding to the gene coding a wild-type catalase protein, and wherein a detected signal from the solid phase is an indication of the absence of the nucleic acid comprising the CAT Exon 8 Leu316Pro (T>C) mutation in the sample.

38. A transgenic animal which carries a human DNA sequence comprising a nucleotide sequence encoding a variant catalase nucleic acid as defined in claim 26.

39. RNA interference methods and models involving a variant nucleotide sequence encoding a variant catalase nucleic acid as defined in claim 26.

40. A method for targeting the treatment of cancer, CHD, and/or stroke according to claim 1, further comprising:
determining the pattern of alleles encoding a catalase; and
treating a subject of the variant genotype with a drug affecting catalase production or metabolism of the subject.

41. The method according to claim 40, wherein the variant genotype is selected from at least one of the group consisting of: −262 C>T of 5′ UTR of the catalase genie, Exon 8 Leu316Pro (T>C) of the catalase gene and Exon 9 Asp389Asp (C>T) of the catalase gene.

42. The method according to claim 40, wherein said variant genotype of the catalase is a homozygote or heterozygote form of mutation.

43. A method for treating a human or animal suffering from cancer, CHD or cerebrovascular stroke or for preventing said disease, said method comprising a therapy enhancing catalase availability, production or concentration of the human subject or animal.

44. The method of claim 43, wherein said animal is a mammal.

45. A method for treating vascular complications of cancer, CHD or stroke, said method comprising a step of enhancing catalase availability, production or concentration in the circulation of a human subject or animal.

46. The method according to claim 43 or 45, said method further comprising administering to a subject a compound enhancing catalase enzyme availability, production or concentration of the subject.

47. The method according to claim 43 or 45, wherein the said method of treating is a dietary treatment or a vaccination.

48. The method according to claim 43 or 45, wherein said therapy is gene therapy or gene transfer.

49. The method according to claim 48, wherein said therapy comprises the transfer of the non-variant catalase gene or fragment or derivative thereof.
